(19) United States
(12) Patent Application Publication
(10) Pub. No.: US 2020/0215065 A1
(43) Pub. Date: Jul. 9, 2020

(54) COMPOSITIONS AND METHOD FOR TREATING PULMONARY HYPERTENSION

(71) Applicant: THE REGENTS OF THE UNIVERSITY OF COLORADO, a body corporate, Denver, CO (US)

(72) Inventors: David IRWIN, Niwot, CO (US); Thies SCHROEDER, Nieder-Olm (DE); Mark BORDEN, Boulder, CO (US); David PAK, Aurora, CO (US); Justin HOPKINS, Thornton, CO (US)

(21) Appl. No.: 16/628,354

(22) PCT Filed: Jul. 6, 2018
(86) PCT No.: PCT/US2018/041133

§ 371 Date: Jan. 3, 2020

Related U.S. Application Data
(60) Provisional application No. 62/530,064, filed on Jul. 7, 2017.

(51) Int. Cl. A61K 31/505; A61K 33/00; A61K 9/00; A61K 9/107; A61P 9/08

(52) U.S. Cl. CPC A61K 31/505; A61K 33/00; A61P 9/08; A61K 9/107; A61K 9/0073

(57) ABSTRACT

Water-in-hydrocarbon emulsions, preferably comprising a fluorinated or perfluorinated hydrocarbon continuous phase, a discontinuous aqueous phase, and a surfactant or mixture of surfactants. The emulsions contain pharmacologically active agents, such as endothelin receptor antagonists, and are particularly suitable for pulmonary drug delivery. The emulsions are useful for treating pulmonary diseases or disorders, including pulmonary hypertension conditions, such as acute pulmonary arterial hypertension.

### CROSS-REFERENCE TO RELATED APPLICATIONS

This application claims priority under 35 U.S.C. § 119(e) to U.S. Provisional Patent Application Ser. No. 62/530,064 filed Jul. 7, 2017, which is incorporated herein in its entirety by reference.

### FEDERALLY SPONSORED RESEARCH OR DEVELOPMENT

This invention was made with government support under Office of Naval Research (ONR) contract No. N00014-16-C-3019 and National Heart, Lung, and Blood Institute (NHLBI) Grants T32HL007171 and 1R01HL125642-01A1. The U.S. government has certain rights in the invention.

### FIELD OF THE INVENTION

The present invention provides compositions and methods useful for treating a subject having a pulmonary hypertension condition.

### BACKGROUND OF THE INVENTION

Pulmonary arterial hypertension (PAH) is a life-threatening and progressive disease of various origins characterized by pulmonary vascular remodeling that leads to increased pulmonary vascular resistance and pulmonary arterial pressure, most often resulting in right-sided heart failure. It is a progressive condition characterized by elevated pulmonary arterial pressures leading to right ventricular (RV) failure. The most common symptom is breathlessness, with impaired exercise capacity being the hallmark of the disease.

PAH is associated with significant morbidity and mortality caused by complex pathways that culminate in structural and functional alterations of the pulmonary circulation and increases in pulmonary vascular resistance and pressure. The progressive narrowing of the pulmonary arterial bed results from an imbalance of vasoactive mediators, including prostacyclin, nitric oxide, and endothelin-1. This leads to an increased right ventricular afterload, right heart failure, and premature death. Diverse genetic, pathological, or environmental triggers, stimulate PAH pathogenesis, culminating in vasoconstriction, cell proliferation, vascular remodeling, and thrombosis.

Besides conservative therapeutic strategies such as anticoagulation and diuretics, drugs approved for the treatment of PAH include inotropic agents (such as digoxin which is a positive inotropic agent that aids in the heart's pumping ability), nifedipine and diltiazem (which act as vasodilators and lower pulmonary blood pressure and may improve the pumping ability of the right side of the heart). In addition to these established therapeutic options, a number of potential therapeutic targets are being investigated, including soluble guanylyl cyclase, phosphodiesterases, tetrahydrobiopterin, 5-hydroxytryptamine (serotonin) receptor 2B, vasoactive intestinal peptide, receptor tyrosine kinases, adrenomedullin, rho kinase, elastases, endogenous steroids, endothelial progenitor cells, immune cells, bone morphogenetic protein and its receptors, potassium channels, metabolic pathways, and nuclear factor of activated T cells.

A promising therapeutic strategy for the treatment of PAH includes endothelin receptor antagonists, which inhibit the upregulated endothelin pathway by blocking the biologic activity of endothelin-1, a mediator responsible for the pathogenesis and progression of PAH. Endothelin receptor antagonists include tezosentan and bosentan, which are dual receptor antagonists affecting both endothelin A and endothlin B receptors, and ambrisentan, sitaxentan, and atrasentan, which affect endothelin A receptors. Ambrisentan is a non-sulfonamide, propanoic acid-class endothelin receptor antagonist (ERA) with high affinity for the endothelin A receptor. Bosentan, a non-selective, sulfonamide-class ERA, is approved for treatment of PAH in patients with WHO functional class III or IV symptoms. Sitaxsentan is another sulfonamide-class ERA that is selective for the endothelin A receptor under review as a PAH therapeutic.

Drug delivery to the distal regions of the lung via inhalational intrapulmonary delivery can be superior for the treatment of lung abnormalities compared to other routes of drug administration such as oral and intravenous (IV) delivery. The feasibility of reaching the pulmonary vasculature with an inhaled drug depends on successful design of the aerosolized delivery vehicle and the method of delivery. Thus, formulation of an effective intrapulmonary drug delivery system is imperative and largely dependent on hydrophobicity, propellant compatibility, stability of the drug carriers, carrier mucoadhesive properties, molecular weight, particle size, and other morphological properties that must be optimized to enhance drug delivery. More specifically, optimum drug-vehicle delivery has been demonstrated for particles in the 1-5 micrometer diameter size range. Smaller and larger particles risk either being exhaled or impacted upon pulmonary branch points preventing dispersion in distal lung regions, respectively. Additionally, alveolar macrophage phagocytosis is greatly reduced for mucoadhesive particles above the 1-5 micrometer size range. Thus, clearance of larger particulates in the lungs is greatly prolonged, which could impede gas exchange in those with pre-existing pulmonary pathologies.

There is a need for stable and effective formulations capable of homogeneous, reproducible pulmonary drug delivery in a controlled manner. The present invention satisfies this need and addresses the requirements of effective intrapulmonary drug delivery system formulations described above.

### SUMMARY OF THE INVENTION

This disclosure provides stable water-in-hydrocarbon emulsions comprising: 1) a continuous (“external” or “bulk medium”) phase comprising 60 to 99.95% (v/v) of at least one hydrophobic hydrocarbon, preferably a fluorinated or perfluorinated organic compound; 2) a discontinuous (“internal”) aqueous phase dispersed in the continuous phase, wherein the discontinuous phase contains a therapeutic agent, and wherein the amount of aqueous phase is between 0.05% and 30% (v/v) of the emulsion; and 3) a surfactant or a mixture of surfactants in the aqueous phase, so that the total amount of surfactant is between 0.01 and 10% (w/v) of the water-in-hydroocarbon emulsion. Preferably, these water-in-hydrocarbon emulsions are water-in-fluorocarbon emulsions, wherein the hydrocarbon is a fluorinated or perfluorinated organic compound.

These emulsions may comprise 80 to 99% (v/v) of the continuous phase; or more preferably, 85 to 95% (v/v) of the continuous phase. The continuous phase may comprise a highly fluorinated compound such as a linear, branched, cyclic, saturated or unsaturated fluorinated hydrocarbon, optionally containing at least one heteroatom and/or bromine or chlorine atom, wherein at least 30% of the hydrogen atoms of said hydrocarbon compound have been replaced by fluorine atoms. Alternatively, or additionally, the emulsion may comprise at least one organic compound that has a fluorinated region and a hydrogenated region. Exemplary continuous phase compounds include dodecane and perfluorooctylbromide (Perflubron; PFOB).

These emulsions may comprise 0.1% to 15% (v/v) of the discontinuous aqueous phase; or more preferably, 1% to 10% (v/v) of the continuous phase. The discontinuous aqueous phase may comprise a biocompatible aqueous solution or suspension comprising at least one therapeutic agent. Exemplary discontinuous aqueous phase media include water, saline, and buffered saline (such as phosphate-buffered saline; PBS).

Surfactants useful in forming the emulsions of this disclosure may be fluorinated surfactants including, for example, amino acid derivatives, amphiphiles containing phosphorus (e.g., perfluoroalkyl or alkylene mono or dimorpholinophosphate and fluorinated phospholipids) or polyhydroxylated or aminated derivatives. Exemplary surfactants include 1,2-diarachidoyl-sn-glycero-3-phosphocholine (DAPC), and Krytox 157 FSH. Exemplary fluorinated surfactants may include (perfluoroalkyl) alkylene dimorpholinophosphate surfactants, such as perfluoroalkylated dimorpholinophosphate (F8H11DMP). Alternatively, or additionally, the emulsion may contain at least one fluorinated surfactant and at least one hydrogenated surfactant. The hydrogenated surfactant may be a phospholipid, polyoxyethylene polyoxypropylene-type copolymer, or polyoxyethylenic sorbitan ester.

The therapeutic agent present in the internal phase may be a water-soluble or water-dispersible pharmacologically active substance. The therapeutically active substance may be a pulmonary vasoactive substance, a mucolytic agent, an antiviral agent, a pharmaceutically active peptide, a nucleic acid, an immunologically active agent, an antibiotic, an antimycobacterial agent, or an anticancer agent. Exemplary therapeutically active agents may include endothelin receptor antagonists selected from tezosentan, bosentan, sitaxentan, ambrisentan, atrasentan, and combinations thereof, and drugs that enhance nitric oxide (NO) production in vivo, such as sodium nitrite.

In addition, the emulsion may further comprise one or more of the following additives: mineral salts, buffer agents, solvents and dispersing agents, oncotic and osmotic agents, nutritive agents, lipophilic pharmacologically active substances. These additional, optional additives may be present in the discontinuous aqueous phase, the continuous phase, at the interface between the phases; or in both of the phases. Preferably, the additive is a water-soluble or water-dispersible pharmacologically active substance present in the discontinuous aqueous phase.

This disclosure also provides processes for the preparation of a water-in-hydrocarbon emulsion comprising a) rehydrating, solubilizing or dispersing a surfactant (such as a fluorinated surfactant) in a discontinuous aqueous phase, optionally containing one or more therapeutically active agents; b) mixing a hydrophobic hydrocarbon (such as a fluorinated or perfluorinated organic compound) continuous phase to the discontinuous phase product of step (a) to form a mixture of hydrocarbon and aqueous phase; and (c) emulsifying the mixture of step (b) to form the water-in-hydrocarbon emulsion.

This disclosure also provides processes for the preparation of a water-in-hydrocarbon emulsion comprising a) rehydrating, solubilizing, or dispersing a surfactant (such as a fluorinated surfactant) in a hydrophobic hydrocarbon (such as a fluorinated or perfluorinated organic compound) continuous phase; b) adding an aqueous phase, optionally containing one or more therapeutically active agents, to the continuous phase product of step (a) to form a mixture of hydrophobic hydrocarbon continuous phase and discontinuous aqueous phase; and (c) emulsifying the mixture of step (b) to form a water-in-hydrocarbon emulsion.

These methods may further comprise the step of sterilizing the emulsion by heat treatment or filtration. Preferably, the emulsifying step (c) is effected by mechanical homogenization, such as in an amalgamator.

Thus, this disclosure provides stable water-in-oil hydrocarbon emulsions comprising a continuous phase comprising 70-99.5% (v/v) of at least one hydrocarbon, preferably a fluorinated or perfluorinated organic compound, a discontinuous aqueous phase dispersed in the continuous phase comprising at least one pharmacologically active agent, wherein the amount of aqueous phase is between 0.05 and 30% (v/v) of the emulsion, and a surfactant, or mixture of surfactants, preferably comprising at least one fluorinated surfactant, wherein the total amount of surfactant is between 0.01 and 10% (w/v) of the emulsion. In these emulsions, the continuous phase may include PFOB or n-dodecane. In these emulsions, the discontinuous phase may include water or PBS. In these emulsions, the fluorinated surfactant may include a (perfluoroalkyl)alkylene dimorpholinophosphate. In these emulsions, the fluorinated surfactant may be at least one of 1,2-diarachidoyl-sn-glycero-3-phosphocholine (DAPC), and perfluoroalkylated dimorpholinophosphate (F8H11DMP). In these emulsions, the at least one pharmacologically active agent may be an endothelin receptor antagonist selected from the group consisting of tezosentan, bosentan, sitaxentan, ambrisentan, and atrasentan. In these emulsions, the at least one pharmacologically active agent may be one or both of ambrisentan and sodium nitrite.

This disclosure therefore provides therapeutic methods for treating a subject exhibiting pulmonary arterial hypertension (PAH), comprising administering to the patient a therapeutically effective amount of an emulsion of this disclosure. Similarly, this disclosure provides a therapeutic method for treating a subject exhibiting hypoxic pulmonary vasoconstriction (HPV), comprising administering to the patient a therapeutically effective amount of an emulsion of this disclosure. In these methods, the HPV may be acute HPV, pulmonary hypertension, elevated pulmonary pressures, or high altitude pulmonary edema.

Similarly, this disclosure provides a therapeutic method for treating a subject exhibiting increased pulmonary arterial pressure (PAP) during exposure to acute systemic hypoxia by administering a therapeutically effective amount of an emulsion of this disclosure to the patient. In these methods, the emulsion is preferably administered by intrapulmonary administration of the emulsion to the patient.

Other embodiments, including particular aspects of the embodiments summarized above, will be evident from the detailed description that follows.

### BRIEF DESCRIPTION OF THE DRAWINGS

FIG. 1A is a schematic representation of the in vivo experimental set up. Hypoxic conditions were induced by mixing room air with N₂ to deliver 13% O₂. The emulsion was administered to the lungs via the endotracheal tube using a Microsprayer®. Mean pulmonary arterial pressure (PAP) and mean systemic arterial pressure (MAP) were measured using fluid-filled indwelling catheters connected to pressure transducers, and recorded with a laptop using the BioPac MP150 system. FIG. 1B depicts the experimental protocol indicating time points for hypoxia (HX), normoxia (NX), and drug delivery throughout the experiment.

FIGS. 2A-2C show sequential images of tested emulsions. Phase separation occurred in DAPC/PFOB (approx. 30 sec) (FIG. 2A) and DAPC/dodecane (approx. 60 sec) (FIG. 2B) emulsion. FIG. 2C is a schematic depiction of the components of the water-in-fluorocarbon emulsion, including the continuous fluorocarbon phase (PFOB), the aqueous discontinuous phase containing the endothelin receptor antagonist ambrisentan or sodium nitrite, and the fluorinated surfactant F8H11DMP. Phase separation rate is significantly reduced when index matching the surfactant and bulk medium as seen with the F8H11DMP/PFOB emulsion (tested over 7 days; FIG. 2C) and the Krytox/PFOB emulsion (also tested over 7 days; FIG. 2D), although the F8H11DMP/PFOB emulsion had better stability than the Krytox/PFOB emulsion. Schematics of formulations and compounds presented with the chemical structures of DAPC, PFOB, dodecane, Krytox and ambrisentan are presented for the phase composition of the emulsion droplets. FIGS. 2E and 2F are brightfield and fluorescent microscopy images of the Krytox/PFOB emulsion for a single and cluster of droplets, respectively, and are <5 micrometers in diameter.

FIGS. 3A and 3B show the mean pulmonary artery pressure (PAP) of intrapulmonary treatments. FIGS. 3C and 3D show the mean systemic arterial pressure (MAP) of intrapulmonary treatments. Measurements were recorded at 2 min intervals and represent mean±SEM. Bar graphs represent the average pressure ±SEM over the 10-min time span of the second bout of hypoxia (HX2). FIGS. 3E and 3F show the mean pulmonary artery pressure (PAP) of intravenous infusion and ambrisentan emulsion. FIGS. 3G and 3H show the mean systemic arterial pressure (MAP) of intravenous infusion and ambrisentan emulsion. SL—Saline; EM—Empty Emulsion; SL+A—Ambrisentan Saline; EM+A—Ambrisentan Emulsion; IV—Intravenous Infusion; NX—normoxic; HX—hypoxic. * p<0.0001

FIGS. 4A-4D show the results of a study conducted to test the use and efficacy of a water-in-fluorocarbon emulsion to encapsulate ambrisentan and administer the emulsion by intrapulmonary drug delivery, using an acute hypoxic rat model monitoring pulmonary arterial pressure, as described in detail in Example 4 of this disclosure. FIG. 4A shows the effect of drug administration on pulmonary arterial pressure. FIG. 4B shows the effect of drug administration on systemic arterial pressure. FIG. 4C shows the effect of drug administration on mean pulmonary arterial pressures in hypoxia.

FIGS. 5A-5E show the results of a study conducted to test the use and efficacy of a water-in-fluorocarbon emulsion to encapsulate sodium nitrite and administer the emulsion by intrapulmonary drug delivery, using an acute hypoxic rat model monitoring pulmonary arterial pressure, as described in detail in Example 5 of this disclosure. FIG. 5A shows the effect of drug administration on pulmonary arterial pressure. FIG. 5B shows the effect of drug administration on systemic arterial pressure. FIG. 5C shows the effect of drug administration on pulmonary arterial pressure. FIG. 5D shows the effect of drug administration on systemic arterial pressure. FIG. 5E shows the effect of drug administration on mean pulmonary arterial pressures in hypoxia.

FIGS. 6A-6C show the results of a study conducted to test the use and efficacy of a water-in-fluorocarbon emulsion to encapsulate the combination of ambrisentan and sodium nitrite, and administer the emulsion by intrapulmonary drug delivery, using an acute hypoxic rat model monitoring pulmonary arterial pressure, as described in detail in Example 6 of this disclosure. FIG. 6A shows the effect of combined drug administration on pulmonary arterial pressure. FIG. 6B shows the effect of combined drug administration on systemic arterial pressure. FIG. 6C shows the effect of combined drug administration on mean pulmonary arterial pressures in hypoxia.

FIGS. 7A-7D show physiological changes to lung evaluated after intrapulmonary dosing of emulsions of this disclosure. FIG. 7A shows macrophage cell count in treated lungs. FIG. 7B shows pulmonary artery pressure 24 hours after the administered doses. FIG. 7C shows mean arterial pressure 24 hours after the administered doses. FIG. 7D shows a histopathology panel of stained lungs 24 hours after administered doses of saline, ambrisentan emulsion, or NaNO₂ emulsion.

### DETAILED DESCRIPTION

The present invention is directed to stable emulsions comprising a continuous hydrocarbon (preferably fluorocarbon) phase into which is dispersed an aqueous phase comprising at least one pharmacologically active agent, and therapeutic methods of using these emulsions. These emulsions may contain hydrophilic or lipophilic therapeutic agents (drugs) and thereby constitute a vehicle for drug administration through the pulmonary route, and possibly other routes of administration, thereby providing homogenous dispersions of a drug in the lungs, and/or other bodies cavities.

### Emulsions and Compositions

Highly fluorinated or perfluorinated organic compounds that may compose the continuous hydrocarbon phase are preferably chosen for their low toxicity, surface tension, spreading coefficient, and/or compatibility with pressurized metered dose inhaler propellants. The use of a surfactant, preferably a fluorinated surfactant, or of a mixture of surfactants comprising at least one fluorinated surfactant, allows the formation of stable water-in-hydrocarbon emulsions. With the use of a fluorinated or perfluorinated organic compound continuous phase, the invention allows the formation of stable water-in-fluorocarbon, or stable water-in-perfluorocarbon, emulsions.

The stable hydrocarbon emulsion may comprise from 60 to 99.95% (v/v) of a hydrophobic continuous phase, preferably made up of a fluorinated or perfluorinated organic compound; from 0.05 to 30% (v/v) of an aqueous phase dispersed in the form of droplets in the continuous phase; and from 0.01 to 10% (w/v) of a surfactant, or a mixture of surfactants, preferably comprising at least one fluorinated surfactant. The volume percentages of the aqueous phase and of the hydrocarbon phase comprise the surfactant or surfactants they contain. In preferred embodiments, these emulsions may contain from 80 to 99% (v/v) of the continuous phase; or more preferably, 85 to 95% (v/v) of the continuous phase.

Fluorinated or perfluorinated compounds useful as the continuous phase of these emulsions may be linear, branched or cyclic, saturated or unsaturated fluorinated hydrocarbons, as well as conventional structural derivatives of these compounds. In addition, these compounds may be totally or partially fluorinated compounds containing one or more heteroatoms, and/or atoms of bromine or chlorine. Partially fluorinated compounds (comprising at least 30% of the hydrogen atoms in the hydrocarbon or derivative thereof replaced with fluorine atoms) are also useful within the continuous phase of these emulsions. Generally, these hydrocarbons comprise from 6 to 20 carbon atoms. Such fluorinated compounds include, but are not limited to, linear, cyclic or polycyclic perfluoroalkanes, perfluoroalkenes, perfluoroamines and perfluoroalkyl bromides. These compounds may be used either alone or in combination. In preferred embodiments, the fluorinated compound consists of perfluorooctyl bromide, C₈F₁₇Br (PFOB), perfluorooctylethane C₈F₁₇C₂H₅ (PFOE).

The continuous fluorocarbon phase may also include a compound having at least one fluorinated region and at least one other hydrogenated region, for example, a linear, branched or cyclic highly fluorinated radical having from about 2 to about 14 carbon atoms and optionally including at least one oxygen atom, and/or at least one halogenated substituent.

The surfactants useful in forming the emulsions of this disclosure are generally strong surfactants, such as 1,2-diarachidoyl-sn-glycero-3-phosphocholine (DAPC) (available commercially from Avanti Polar Lipids; Alabaster, Ala.). The surfactants may be hydrogenated, non-ionic, anionic, cationic or zwitterionic surfactants. Useful hydrogenated surfactants include, for example, phospholipids, copolymers of the polyoxyethylene polyoxypropylene type (e.g., Pluronic F-68®). and polyoxyethylene sorbitan esters.

The surfactants may contain fluorine atoms, i.e., fluorinated surfactants that may be of different types such as amino acid derivatives, amphiphiles containing phosphorus (e.g., (perfluoroalkyl) alkylene mono or dimorpholinophosphate and fluorinated phospholipids) or polyhydroxylated or aminated derivatives including amine oxides. Exemplary fluorinated surfactants include perfluoroalkylated dimorpholinophosphates, such as perfluoroalkylated dimorpholinophosphate (F8H11DMP).

The emulsions of the invention also comprise a pharmacologically active substance dispersed in the aqueous (discontinuous) phase of the emulsion. Examples of useful pharmacologically active substances include endothelin-1 receptor antagonist compounds such as tezosentan, bosentan, sitaxentan, ambrisentan, and/or atrasentan; drugs that enhance nitric oxide (NO) production in vivo, such as sodium nitrite; antibiotics such as gentamicin, erythromycin, and doxycycline; tuberculostatic antimycobacterials such as pyrazinamide, ethambutol, and isoniazid; anticancer agents such as cisplatin, cyclophosphamide, 5-fluorouracil, and doxorubicin; pulmonary vasoactive substances and regulators of pulmonary hypertension such as tolazoline; respiratory stimulants such as doxapram; vasoactive bronchodilators such as epinephrine and theophylline; mucolytic agents such as acetylcysteine; antiviral agents such as ribavirin; and surfactants such as dipalmitoylphosphatidylcholine.

These emulsions may also comprise one or more additives which are present either in the dispersed aqueous phase, or in the continuous hydrorocarbon phase, in both of these phases, or at the interface between the phases. The additives may include, for example, mineral salts, buffers, oncotic and osmotic agents, nutritive agents, active principles, the pharmacologically active substances described above, nucleic acids, genetic material, immunoactive agents, or any other ingredient capable of augmenting the favorable characteristics of the emulsions including their stability, therapeutic efficacy, tolerance or compatibility with other formulation ingredients, such as pressurized metered dose inhaler propellants.

The emulsions of this disclosure are generally prepared by solubilizing or dispersing the surfactant, or mixture of surfactants, in the aqueous (discontinuous) phase by mechanical stirring; adding the appropriate quantity of continuous (hydrophobic) phase, which can contain one or more surfactants, dispersant agents, and/or additives, to the aqueous phase to form a mixture.

Alternatively, the emulsions of this disclosure may be prepared by solubilizing or dispersing the surfactant, or mixture of surfactants, in the hydrophobic, hydrocarbon (continuous) phase by mechanical stirring; adding the appropriate quantity of aqueous (discontinuous) phase, which may contain one or more surfactants, dispersant agents, and/or additives to the continuous, hydrophobic phase to form a mixture.

In either method, the mixture is then emulsified by conventional homogenization such as, amalgamation, microfluidization, sonication, and/or homogenization under pressure.

The emulsions of the invention may be sterilized, for example, by autoclaving, or by filtration, for example through a 0.22-micron filter.

These emulsions can also be diluted in another hydrocarbon, such as a fluorocarbon, to adjust concentration, dosage, or administration regimen.

### Therapeutic Methods

Pulmonary hypertension may be mild, moderate or severe, as measured for example by WHO functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment. Four functional classes are recognized in the WHO system:

Class I: pulmonary hypertension without resulting limitation of physical activity; ordinary physical activity does not cause undue dyspnea or fatigue, chest pain or near syncope;

Class II: pulmonary hypertension resulting in slight limitation of physical activity; patient comfortable at rest; ordinary physical activity causes undue dyspnea or fatigue, chest pain or near syncope;

Class III: pulmonary hypertension resulting in marked limitation of physical activity; patient comfortable at rest; less than ordinary activity causes undue dyspnea or fatigue, chest pain or near syncope;

Class IV: pulmonary hypertension resulting in inability to carry out any physical activity without symptoms; patient manifests signs of right-heart failure; dyspnea and/or fatigue may be present even at rest; discomfort is increased by any physical activity.

As used herein, “treatment” may encompass: (a) adjustment of one or more hemodynamic parameters towards a more normal level, for example lowering mean PAP or PVR, or raising PCWP or LVEDP, versus baseline; (b) improvement of pulmonary function versus baseline, for example increasing exercise capacity, (for example as measured in a test of 6-minute walking distance (6MWD), or lowering Borg dyspnea index (BDI)); (c) improvement of one or more quality of life parameters versus baseline, for example an increase in score on at least one of the health survey functional scales; (d) general improvement versus baseline in the severity of the condition, for example by movement to a lower WHO functional class; (e) improvement of clinical outcome following a period of treatment, versus expectation in absence of treatment (e.g., in a clinical trial setting, as measured by comparison with placebo), including improved prognosis, extending time to or lowering probability of clinical worsening, extending quality of life (e.g., delaying progression to a higher WHO functional class or slowing decline in one or more quality of life parameters such as in health survey parameters), and/or increasing longevity; and/or (f) adjustment towards a more normal level of one or more molecular markers that can be predictive of clinical outcome (e.g., plasma concentrations of endothelin-1 (ET-1), cardiac troponin T (cTnT) or B-type natriuretic peptide (BNP)).

A “therapeutically effective amount” of an endothelin receptor antagonist, such as ambrisentan, is an amount (typically a daily amount administered over the course of a period of treatment) sufficient to provide any one or more of the effects mentioned above. Preferably, the amount administered does not exceed an amount causing an unacceptable degree of adverse side effects. What constitutes a therapeutically effective amount can vary depending on the particular pulmonary hypertension condition to be treated, the severity of the condition, body weight and other parameters of the individual subject, and can be readily established without undue experimentation by the physician or clinician based on the disclosure herein. Such amount can be administered each day, for example in individual doses administered once, twice, or three or more times a day. However, dosages stated herein on a per day basis should not be construed to require administration of the daily dose each and every day. For example, if ambrisentan is provided in the emulsion, daily dosage amounts may be administered at a lower frequency, e.g., every second day to once a month, or even longer. Most typically and conveniently for the patient, ambrisentan is administered once a day, for example in the morning.

The pharmacologically active agent may be administered for an extended treatment period. Typically, the longer the treatment continues, the greater and more lasting will be the benefits. Illustratively, the treatment period can be at least about one month, for example at least about 3 months, at least about 6 months or at least about 1 year. In some cases, administration can continue for substantially the remainder of the life of the subject.

The emulsions of this disclosure may be administered by any suitable route including intrapulmonary (e.g., by inhalation) route. Oral administration may also be contemplated for some subjects and can occur independently of meal times, i.e., with or without food.

The subject treated with the emulsions of this disclosure may experience, during or following the treatment period, at least one of (a) adjustment of one or more hemodynamic parameters indicative of the pulmonary hypertension condition towards a more normal level versus baseline; (b) increase in exercise capacity versus baseline; (c) lowering of BDI versus baseline; (d) improvement of one or more quality of life parameters versus baseline; and/or (e) movement to a lower WHO functional class. Any suitable measure of exercise capacity can be used; a particularly suitable measure is obtained in a 6-minute walk test (6MWT), which measures how far the subject can walk in 6 minutes, i.e., the 6-minute walk distance (6MWD). The Borg dyspnea index (BDI) is a numerical scale for assessing perceived dyspnea (breathing discomfort). It measures the degree of breathlessness after completion of the six-minute walk test (6MWT), where a BDI of 0 indicates no breathlessness and 10 indicates maximum breathlessness.

In an exemplary aspect, the pharmacologically active agent administered within the emulsions of this disclosure is an endothelin inhibitor, such as ambrisentan, that is administered in an amount effective to adjust one or more hemodynamic parameters indicative of the pulmonary hypertension condition towards a more normal level. In one such aspect, mean PAP is lowered, for example by at least about 3 mmHg, or at least about 5 mmHg, versus baseline. In another such aspect, PVR is lowered. In yet another such aspect, PCWP or LVEDP is raised.

In an exemplary aspect, the endothelin inhibitor, such as ambrisentan, can be administered in an amount effective to improve pulmonary function versus baseline. Any measure of pulmonary function can be used; illustratively 6MWD is increased or BDI is lowered. 6MWD may be increased from baseline by at least about 10 m, for example at least about 20 m or at least about 30 m. In many instances, the method will be effective to increase 6MWD by as much as 50 m or even more.

In an exemplary aspect, the endothelin inhibitor, such as ambrisentan, can be administered in an amount effective to improve quality of life of the subject, illustratively measured by one or more of the health parameters recorded in an SF-36 survey. For example, an improvement versus baseline is obtained in at least one of the SF-36 physical health related parameters (physical health, role-physical, bodily pain and/or general health) and/or in at least one of the SF-36 mental health related parameters (vitality, social functioning, role-emotional and/or mental health). Such an improvement can take the form of an increase of at least 1, for example at least 2, or at least 3 points, on the scale for any one or more parameters.

The endothelin inhibitor, such as ambrisentan, can be administered in alone or in combination therapy with one or more additional drugs. For example, the endothelin inhibitor, such as ambrisentan, can be administered in combination therapy with a second active agent effective for the treatment of the pulmonary hypertension condition or a condition related thereto. When the endothelin inhibitor, such as ambrisentan, is administered concomitantly, one of skill in the art can readily identify a suitable dose for any particular second active agent. Illustratively and without limitation, the endothelin inhibitor, such as ambrisentan, can be administered with a second active agent comprising at least one drug selected from the group consisting of prostanoids, phosphodiesterase inhibitors (especially phosphodiesterase-5 (PDES) inhibitors), additional, other endothelin receptor antagonists (ERAs), such as ERAs other than ambrisentan, calcium channel blockers, diuretics, anticoagulants, oxygen, and combinations thereof.

Examples of drugs useful in combination therapy with endothelin inhibitor, such as ambrisentan, are drugs active at more than one target, such as another pulmonary receptor. Accordingly, use of any such drug in a combination is contemplated herein, independently of its mode of action.

### EXAMPLES

The following examples are merely illustrative, and do not limit this disclosure in any way. Reference is made in the examples to statistical analysis and statistical significance of results. Such reference is made in the interest of full disclosure and does not constitute admission that statistical significance is a prerequisite for patentability of any claim herein.

Materials Used in these Examples

Emulsions were prepared using 1,2-diarachidoyl-sn-glycero-3-phosphocholine (DAPC) (Avanti Polar Lipids; Alabaster, Ala.) or Krytox 157 FSH (DuPont; Wilmington, Del.) as the surfactants, and the bulk, continuous, medium phase was composed of PFOB (Fluoromed, L.P.; Round Rock, Tex.) or n-dodecane (Sigma-Aldrich; St. Louis, Mo.). The water phase was made up of phosphate-buffered saline (PBS; 0.1 M NaCl) with a pH of 7.6, and ambrisentan (Duke Small Molecule Synthesis Facility; Durham, N.C.) was dissolved in the PBS. The components of the emulsion were placed in 2 mL serum vials and emulsified using the D650 Amalgamator (TPC Advanced Technologies; City of Industry, CA) at a rate of 4,400 RPM for two 40-second intervals. All formulations were prepared as water-in-oil emulsions, and will be referred to by the surfactant and continuous phases for the remainder of these Examples. Adult male Sprague-Dawley rats (n=42) (Charles River; Wilmington, Mass.) were allowed ad libitum access to food and water and were kept on a 12-h day-night cycle.

Statistical Analyses Used in these Examples

Statistical comparisons for data measurements were completed using one-way analysis of variance (ANOVA) with the Tukey correction for multiple comparisons. Post-hoc analyses were completed with unpaired, two-sided Student's t-test. Statistical analyses were performed using GraphPad Prism (Version 6) statistical software package (Graphpad Software, Inc; La Jolla, Calif.) with statistical significance set at p<0.05. For all groups, mean±S.E.M is reported.

### Example 1

### Emulsion Preparation

A. DAPC/PFOB and DAPC/Dodecane

The DAPC/PFOB and DAPC/dodecane emulsions were prepared by rehydrating DAPC in PBS at a concentration of 1% w/v. The solution was heated to 60° C. and mixed with a magnetic stir bar for 10 minutes. The lipid solution was cooled to 25° C. and added to the bulk medium at a volume ratio of 1:9 v/v, respectively. The mixture was then emulsified using the amalgamator.

B. Krytox/PFOB

The Krytox/PFOB emulsions were prepared by first adding Krytox dropwise to PFOB to a final surfactant concentration of 9% w/v concentration. The PBS was then added to the Krytox/PFOB solution 1:9 v/v and emulsified using the amalgamator.

### Example 2

### Emulsion Characterization

To characterize emulsion stability, immediately following emulsification, the mixtures in the vials were recorded and observed for any characteristic changes over a 7-day period. Additionally, fluorescent isothiocyanate-dextran (MW: 70 kg mol-1) (Sigma-Aldrich; St. Louis, Mo.) was solubilized into the PBS of the Krytox/PFOB emulsion, and imaged with the Olympus BX52 (Olympus; Center Valley, Pa.) fluorescent microscope using 492 nm and 518 nm excitation and emission wavelengths, respectively. The emulsion was diluted 1:99 v/v with PFOB, and pipetted onto slides with a coverslip to prevent evaporation. Images were captured with the QIClick CCD Camera (QImaging; Surrey, BC, Canada) and processed using Image J (Version 1.8) (National Institute of Health; Bethesda, Md.; imagej.nih.gov/ij/). The contrast and brightness thresholds of the images were adjusted to clearly depict the droplets in the bulk medium.

For the ambrisentan encapsulation in Krytox droplets: For the in vivo studies, the pH of the PBS was raised to 7.8 in order to increase the ambrisentan concentration in the emulsion, and resulted in a solubility of approximately 100 mg/mL ambrisentan in PBS. The aqueous solution of ambrisentan was then mixed with Krytox and PFOB and emulsified as previously described.

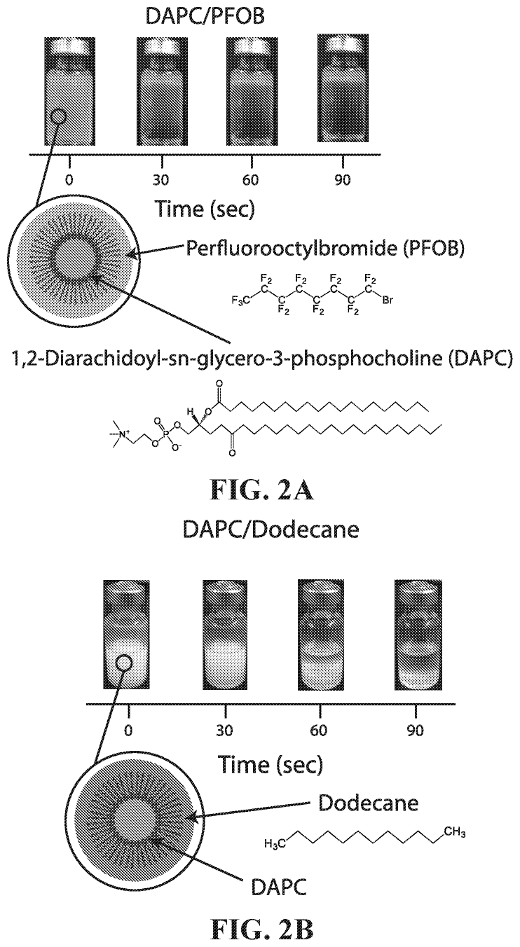

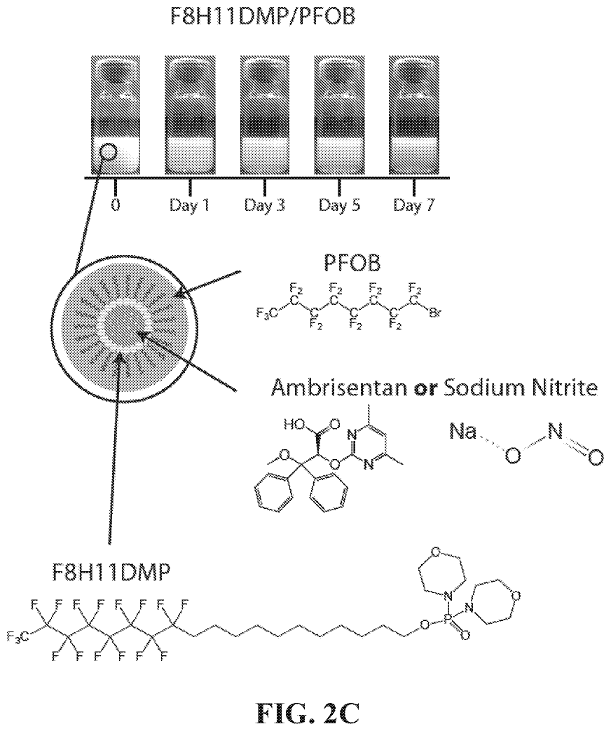

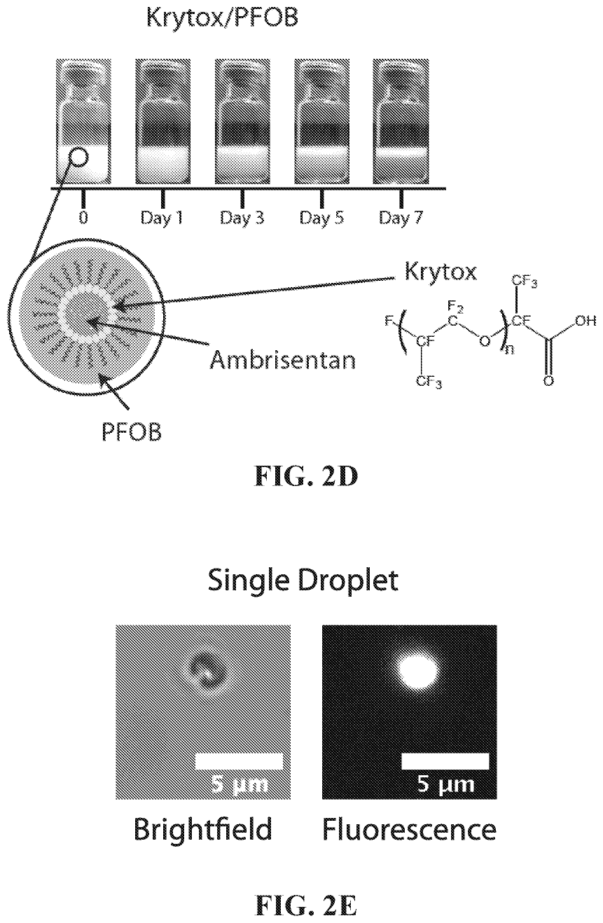

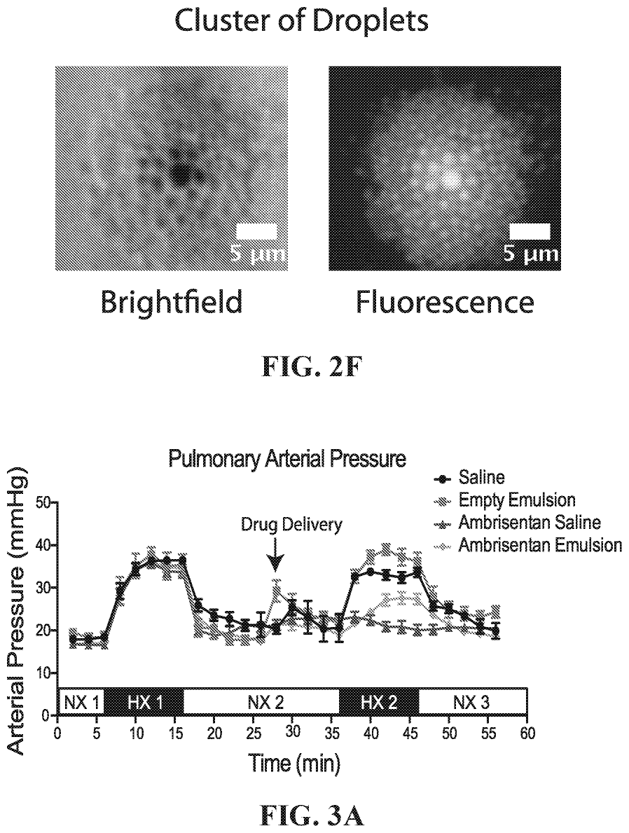

FIGS. 2A-2C show the effect of emulsion composition on phase separation. Immediately after the emulsification process, all three formulations appeared as a milky white solution. Phase separation was evident as creaming of the white aqueous droplets above the transparent PFOB phase. The greatest stability was observed for the F8H11DMP/PFOB formulation (FIG. 2C). Fluorescent microscopy confirmed the water-in-fluorocarbon composition and showed that emulsion droplet diameters ranged from 1 to 5 microns (FIGS. 2E and 2F).

### Example 3

### In Vivo Emulsion Testing

Surgical procedures and hemodynamic measurements: Rats were anesthetized by intramuscular injection with ketamine/xylazine mixture (75 and 6 mg/kg, respectively). The ventral neck was shaved and a 2 cm incision was made in the right ventral neck where the jugular vein and right carotid artery were isolated via blunt dissection. A polyethylene (PE-50) catheter was introduced into the right carotid to measure systemic blood pressure. A polyvinyl (PV-1) catheter was inserted into the jugular vein and threaded through the right atrium, right ventricle, and into the lumen of the main pulmonary artery to obtain pulmonary arterial pressure. Fluid filled catheters were connected to pressure transducers and monitored continuously with the MP150 data acquisition system (BioPAC Systems; Goleta, Calif.). Blood pressures were recorded every two minutes for data analysis.

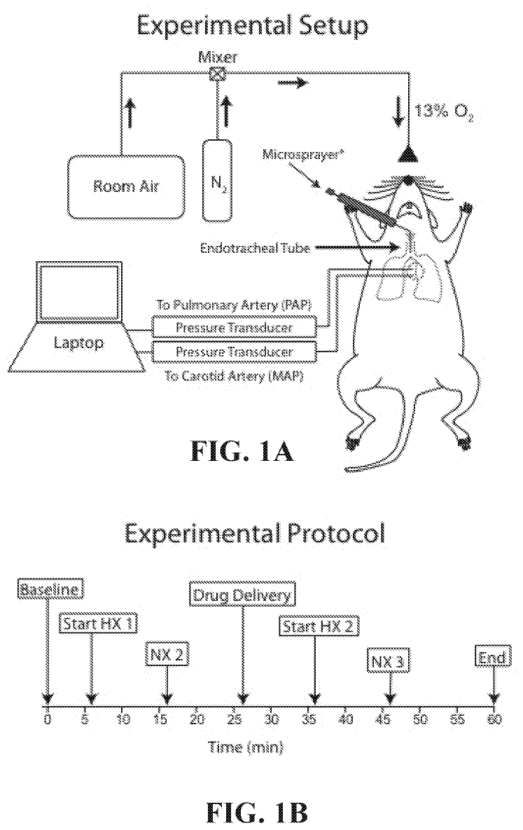

After completion of catheter placements and instrumentation for hemodynamic measurements, a 1 cm incision was made above the trachea, and the trachea was isolated by blunt dissection. A tracheotomy was performed approximately 4 mm above the carina and an endotracheal tube specially designed for rats was inserted such that the end of the tube was placed roughly 2 mm distal from the carina. This placement allowed the tip of a Microsprayer® attached to the FMJ-250 syringe (Penn-Century; Philadelphia, Pa.) to protrude approximately 1 mm from the trachea tube and deliver aerosolized emulsion to both right and left lung periphery (FIG. 1A).

Testing efficacy of intrapulmonary delivery of ambrisentan: following instrumentation and tracheotomy, anesthetized rats were placed in a specially designed Plexiglas box built to accommodate exteriorized catheters for completion of the study protocol (depicted graphically in FIG. 1B). Baseline PAP and mean systemic arterial pressure (MAP) were recorded. Next, rats were switched from breathing room air to a hypoxic gas mixture for 10 minutes. Acute hypoxia was induced in rats by exposure to 13% O₂ (room air/nitrogen dilution; FIG. 1A). This model previously demonstrated that acute hypoxia induced the HPV-mediated response that raised pulmonary arterial pressures. This first hypoxic challenge was followed by 20 minutes of room air breathing. The purpose of this initial bout of hypoxic air breathing was to confirm that each animal had an intact HPV-mediated rise in PAP. During this period of room air breathing, rats were allowed to recover from hypoxia for 10 minutes prior to treatment administration via the Microsprayera Treatments were delivered as a 100-microliter bolus aerosol immediately following emulsification to avoid potential phase separation. Following treatment, rats remained breathing room air for an additional 10 minutes to allow drug absorption before being exposed to a second 10-minute hypoxic challenge. Following the second hypoxic challenge, rats returned to breathing room air and blood pressures were monitored for 15 minutes before animals were euthanized with intravenous injection of a euthanasia agent.

Intravenous infusion: As a reference to aid in understanding the response of intrapulmonary delivery of ambrisentan, an additional treatment group for intravenous (IV) infusion of ambrisentan was also investigated (5 mg/kg; 0.5 mL saline). For this treatment group of rats, an additional PV-1 catheter was inserted in the femoral vein to administer ambrisentan to avoid interrupting hemodynamic measurements.

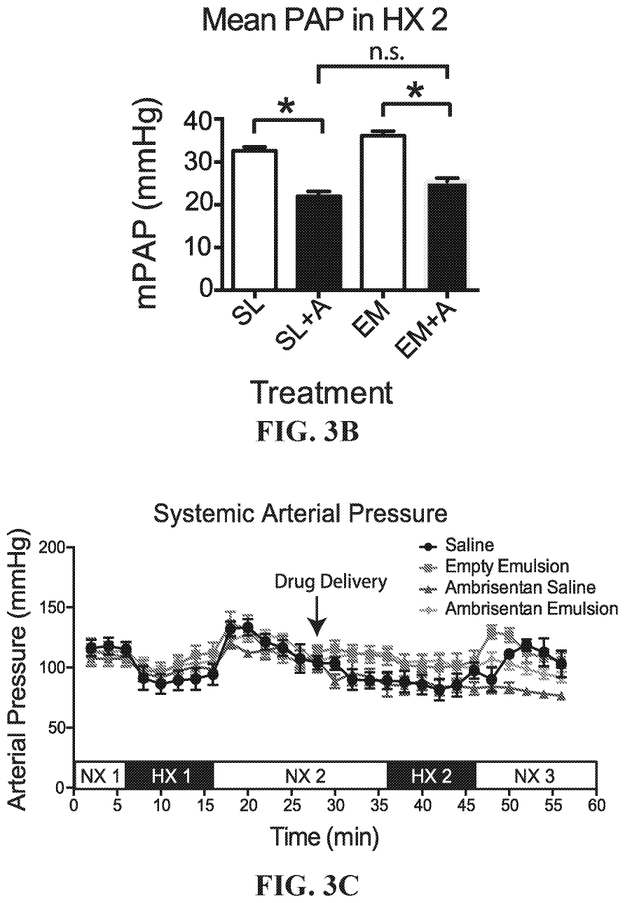

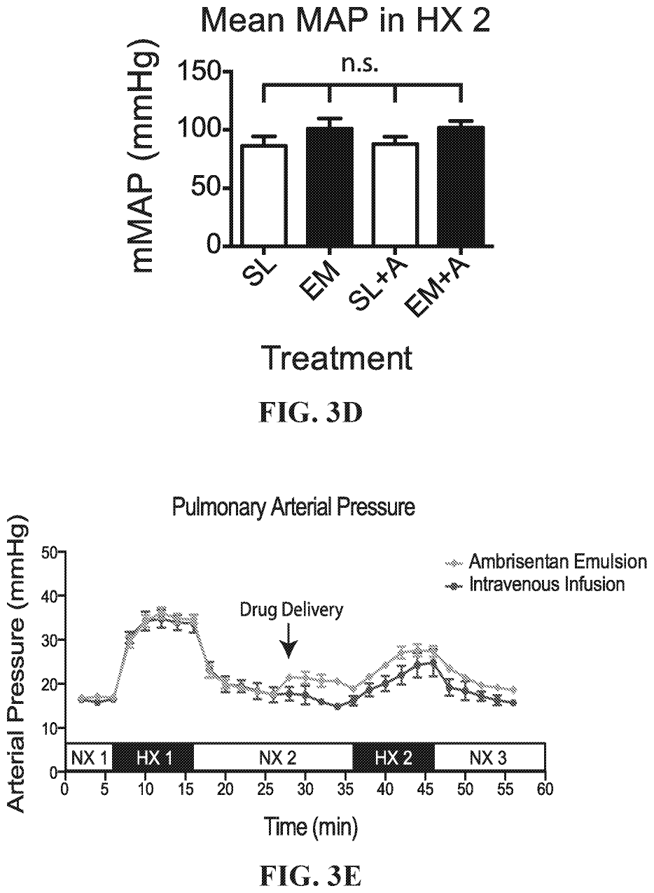

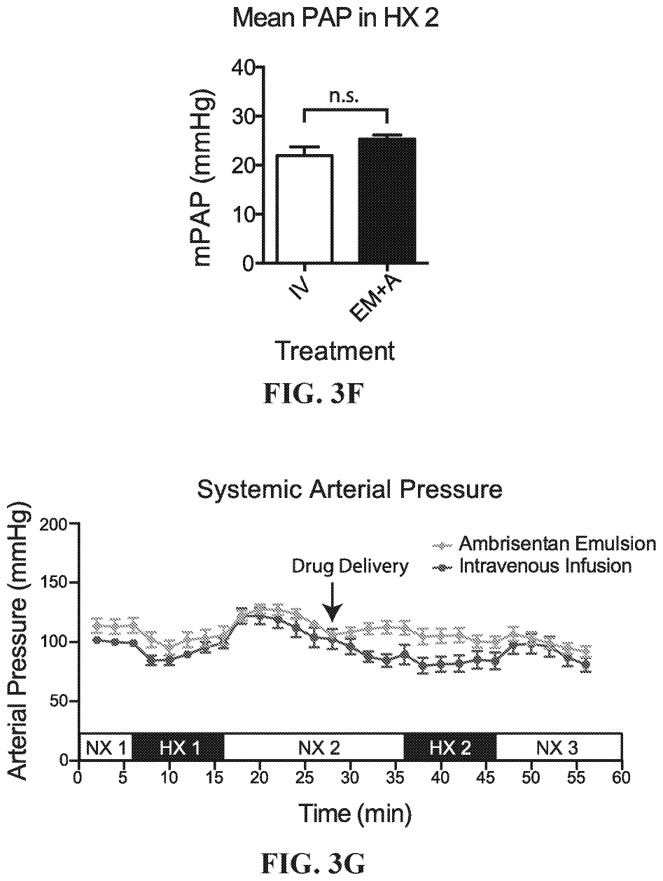

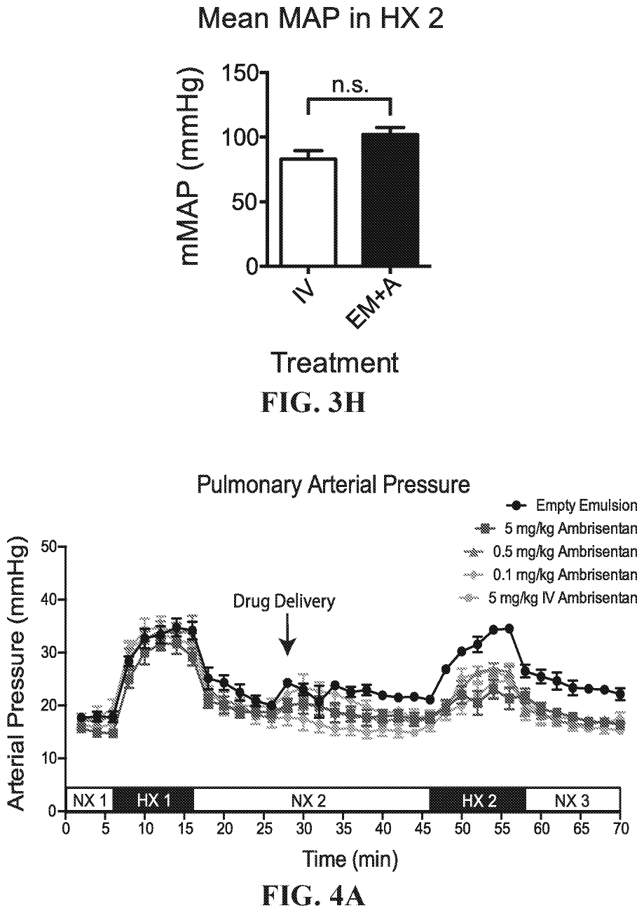

Baseline mean PAP and MAP were similar among all rats across cohorts and confirmed normal healthy animals (FIG. 3A). We observed the expected percent increase in PAP (94.68±4.28%) and fall in MAP (18.46±3.12%) after challenging rats with hypoxic air. Both PAP and MAP returned to baseline values within 10 minutes once rats returned to room air breathing.

All rats tolerated the intrapulmonary delivery of the vehicle or emulsions through the endotracheal tube with no significant changes in either PAP, MAP, or breathing and heart rates. During the second hypoxic challenge, the rise in PAP was significantly reduced in rats receiving ambrisentan, regardless of the delivery vehicle, compared to rats that received either saline or empty emulsion (FIGS. 3A and 3B). When referenced to the IV administration, we observed a similar inhibition (FIGS. 3E and 3F) and confirmed that ambrisentan was delivered through the intrapulmonary route. In contrast to PAP, our data showed that ambrisentan did not further reduce the MAP during the second hypoxic challenge (FIGS. 3C and 3D) which also held true with IV administration (FIGS. 3G and 3H).

The data presented in these Examples, demonstrate that drug encapsulation using the water-in-fluorocarbon emulsion yielded a stable formulation for intrapulmonary delivery of an endothelin receptor antagonist, ambrisentan. Further, the studies exploiting the HPV response demonstrate this delivery method is an effective means for targeting pulmonary vascular diseases, such as pulmonary hypertension (PH).

### Example 4

### Aerosolized Administration of Ambrisentan on Attenuating the HPV Response

This study was conducted to test the use and efficacy of a water-in-fluorocarbon emulsion of this disclosure to encapsulate ambrisentan or sodium nitrite for intrapulmonary drug delivery. An acute hypoxic rat model was used, and pulmonary and systemic arterial pressure were recorded to determine efficacy of the drug delivery system for treatment of the acute hypoxic pulmonary vasoconstrictive response.

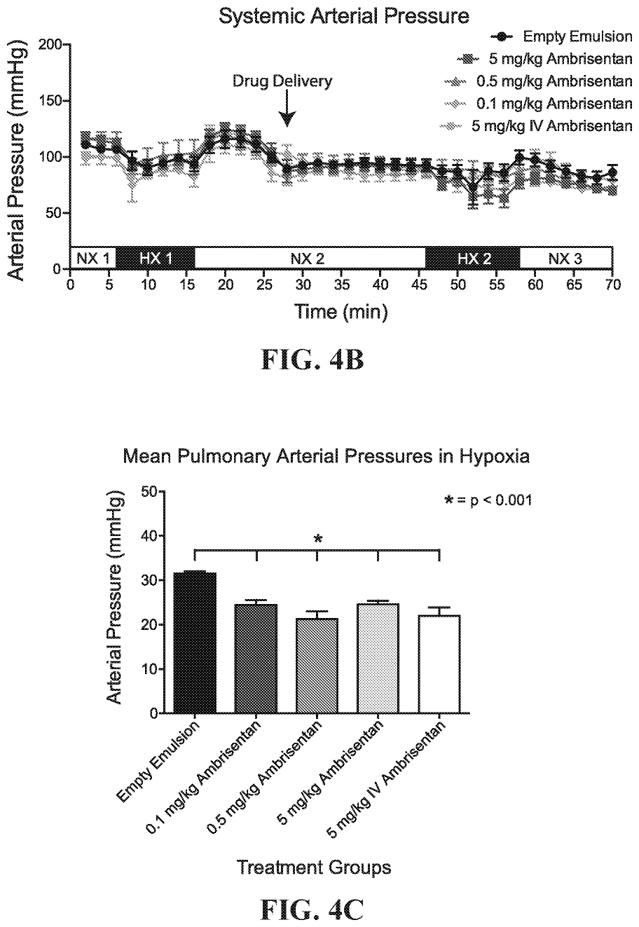

Rats were randomized to three groups: (1) high dose ambrisentan (5 mg/kg); (2) mid dose (0.5 mg/kg); and (3) low dose (0.1 mg/kg) (n=6 per group). As shown in FIGS. 4A-4C, intrapulmonary drug delivery of ambrisentan at various doses significantly reduced the mean pulmonary arterial pressure in rats when exposed to acute hypoxia at all doses (FIG. 4A). The intrapulmonary drug delivery system was not significantly different than the response after rats received intravenous infusion of ambrisentan (FIGS. 4A and 4C). Additionally, the systemic arterial pressure had the expected fluctuation when exposed to acute hypoxia, and no adverse effects were observed (FIG. 4B).

### Example 5

### Aerosolized Administration of Sodium Nitrite (NaNO₂) on Attenuating the HPV Response

This study was conducted to test the use and efficacy of a water-in-fluorocarbon emulsion to encapsulate sodium nitrite for intrapulmonary drug delivery. An acute hypoxic rat model was used, and pulmonary and systemic arterial pressure were recorded as a means to determine efficacy of the drug delivery system for treatment of the acute hypoxic pulmonary vasoconstrictive response.

Rats were randomized to two groups: (1) high dose sodium nitrite (0.5 mg/kg); and (2) low dose (0.1 mg/kg); (n=2 per group).

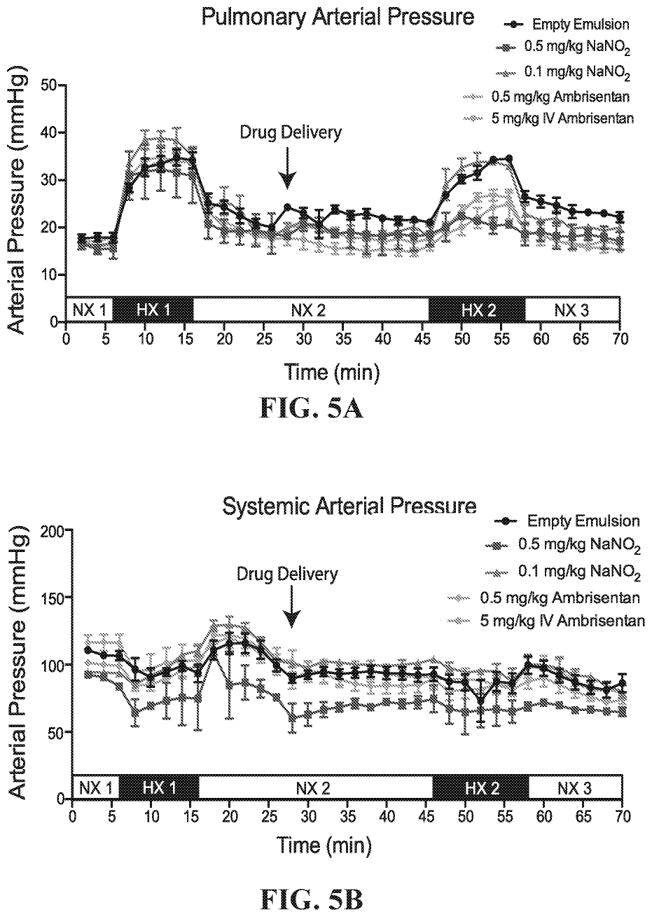

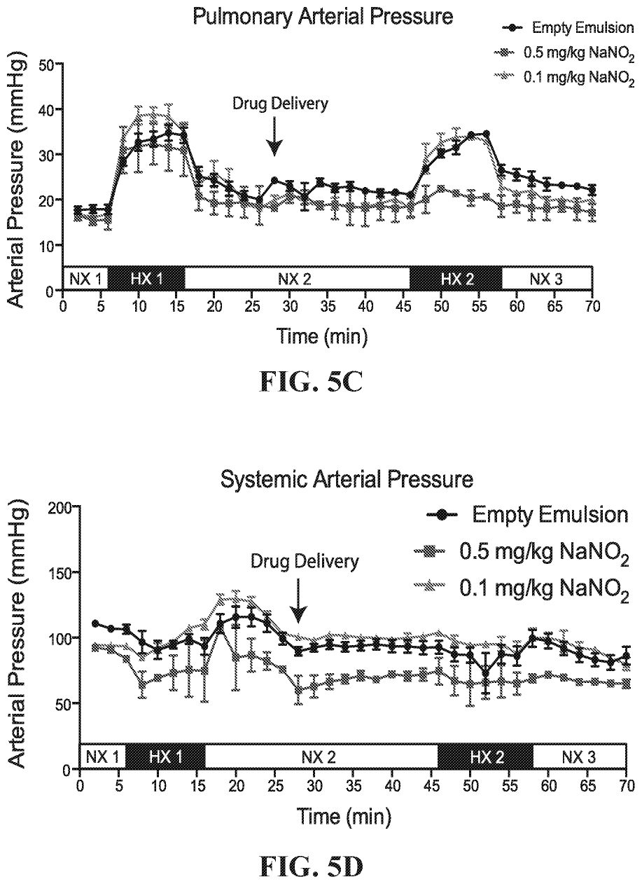

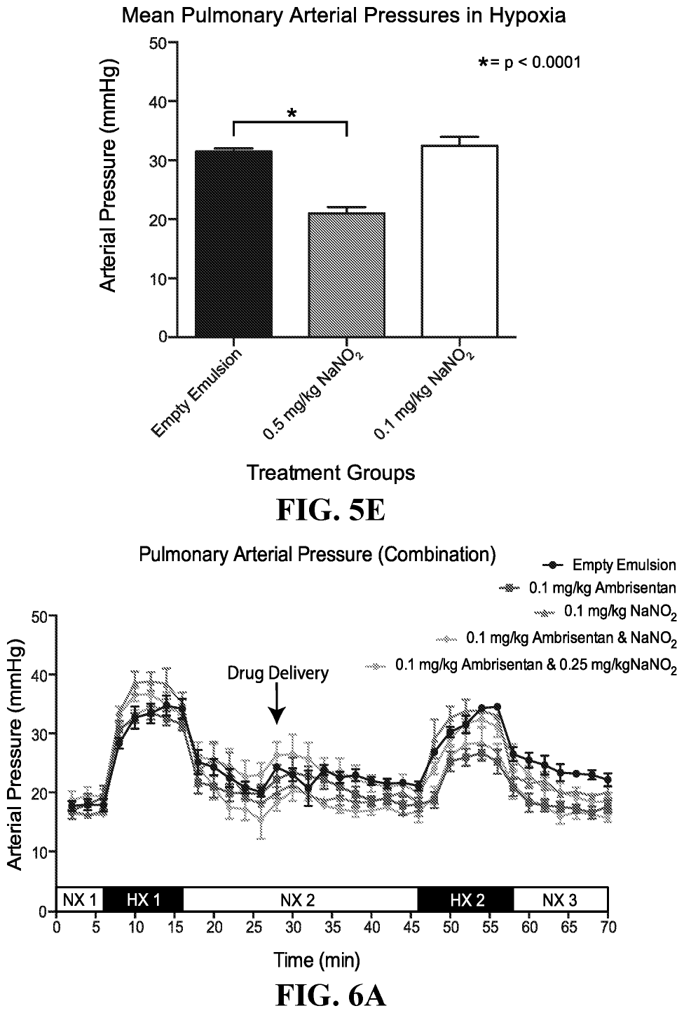

As shown in FIGS. 5A, 5C, and 5E, intrapulmonary drug delivery of the high dose sodium nitrite at various doses significantly reduced the mean pulmonary arterial pressure in rats when exposed to acute hypoxia. However, when dosed with 0.1 mg/kg sodium nitrite, the pulmonary arterial pressure did not significantly change compared to the empty emulsion (FIG. 5E). Additionally, the systemic arterial pressure had the expected fluctuation when exposed to acute hypoxia, and no adverse effects were observed (FIGS. 5B and 5D).

### Example 6

### Aerosolized Administration of Ambrisentan Combined with Sodium Nitrite (NaNO₂) Attenuate the HPV Response

This study was conducted to test the use and efficacy of a water-in-fluorocarbon emulsion to encapsulate both ambrisentan and sodium nitrite for intrapulmonary drug delivery, and to study how this combination therapy affected the HPV response. An acute hypoxic rat model was used, and pulmonary and systemic arterial pressure were recorded as a means to determine efficacy of the drug delivery system for treatment of the acute hypoxic pulmonary vasoconstrictive response.

Rats were randomized to two groups: (1) mid dose combination (0.1 mg/kg) ambrisentan, and 0.25 mg/kg sodium nitrite); and (2) low dose combination (0.1 mg/kg). (n=4 per group).

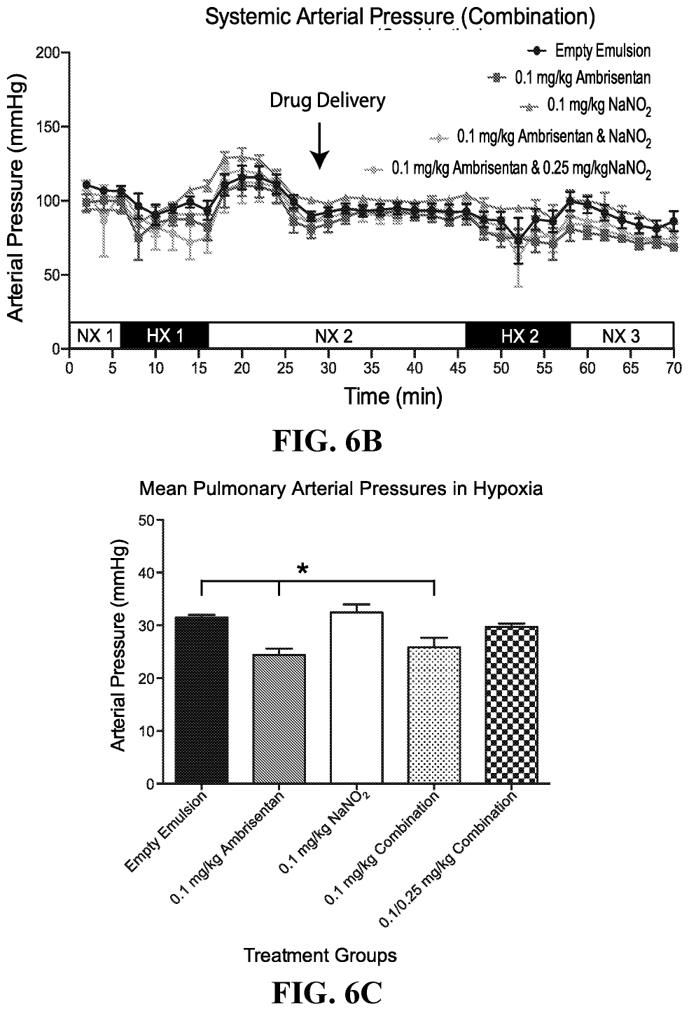

As shown in FIGS. 6A and 6C, intrapulmonary drug delivery of the combination at various doses significantly reduced the mean pulmonary arterial pressure in rats when exposed to acute hypoxia. The intrapulmonary drug delivery system had a similar effect as ambrisentan alone with the low dose combination, and did not have a significant effect with the mid dose (FIGS. 6A and 6C). Additionally, the systemic arterial pressure had the expected fluctuation when exposed to acute hypoxia, and no adverse effects were observed (FIG. 6B).

### Example 7

### Irritation and Toxicity Testing of Aerosol in Lung

This study was conducted to evaluate physiological changes to the lungs of test animals after administration of a water-in-fluorocarbon emulsion to encapsulate both ambrisentan and sodium nitrite for intrapulmonary drug delivery. The acute hypoxic rat model described above was used, and lungs were harvested and inflated 24 hours after administered dose for saline, ambrisentan emulsion (5 mg/kg), and NaNO₂ emulsion (5 mg/kg).

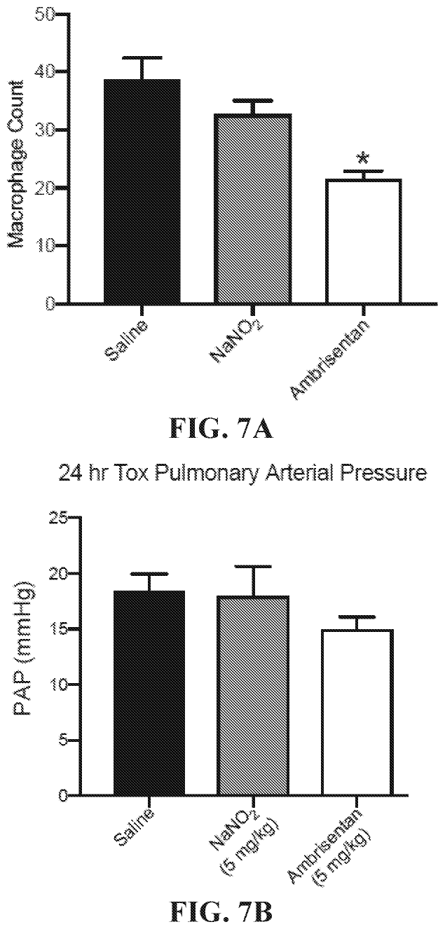

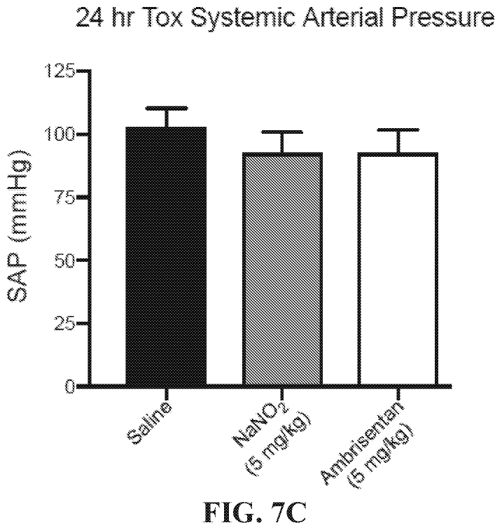

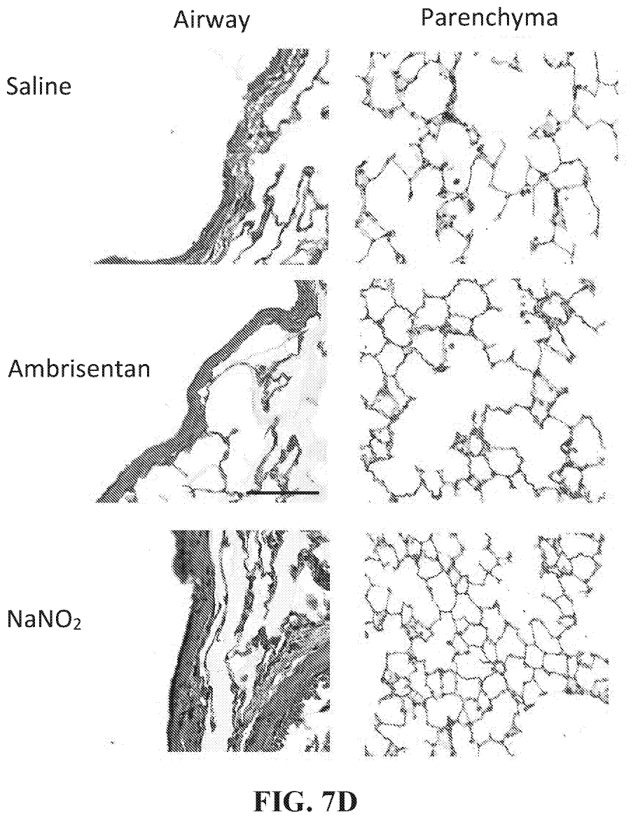

FIGS. 7A-7D show physiological changes after intrapulmonary dosing. FIG. 7A shows macrophage cell count per frame (* p<0.05) in treated lung. FIG. 7B shows pulmonary artery pressure 24 hours after administered dose. FIG. 7C shows mean arterial pressure 24 hours after administered dose. FIG. 7D shows a histopathology panel of H&E stained lungs harvested and inflated 24 hours after administered dose for saline, ambrisentan emulsion (5 mg/kg), and NaNO₂ emulsion (5 mg/kg).

Varying substitutions and modifications may be made to the invention disclosed herein without departing from the spirit of the invention. The present invention has been specifically disclosed by the preferred modification and variation of the concepts herein disclosed may be resorted to by those skilled in the art, and such modifications and variations are considered to be falling within the scope of the invention. The phraseology and terminology used herein is for the purpose of description and should not be regarded as limiting. The use of “including,” “comprising,” or “having” and variations thereof herein is meant to encompass the items listed thereafter and equivalents thereof as well as additional items.

1. A stable water-in-hydrocarbon emulsion, comprising:
a continuous phase comprising 70-99.5% (v/v) of at least one hydrocarbon;
a discontinuous aqueous phase dispersed in the continuous phase comprising at least one pharmacologically active agent, wherein the amount of aqueous phase is between 0.05% and 30% (v/v) of the emulsion; and
a surfactant, or mixture of surfactants, comprising at least one fluorinated surfactant, wherein the total amount of surfactant is between 0.01 and 10% (w/v) of the emulsion.

2. The emulsion of claim 1, wherein the continuous phase hydrocarbon is a fluorinated or perfluorinated organic compound.

3. The emulsion of claim 1, wherein the continuous phase comprises PFOB or n-dodecane.

4. The emulsion of claim 1, wherein the discontinuous phase comprises water or phosphate-buffered saline PBS.

5. The emulsion of claim 1, wherein the fluorinated surfactant is a (perfluoroalkyl)alkylene dimorpholinophosphate.

6. The emulsion of claim 1, wherein the fluorinated surfactant comprises at least one of 1,2-diarachidoyl-sn-glycero-3-phosphocholine (DAPC) and perfluoroalkylated dimorpholinophosphate (F8H11DMP).

7. The emulsion of claim 1, wherein the at least one pharmacologically active agent is an endothelin receptor antagonist selected from the group consisting of ambrisentan, tezosentan, bosentan, sitaxentan, and atrasentan.

8. The emulsion of claim 1, wherein the at least one pharmacologically active agent is one or both of ambrisentan and sodium nitrite.

9. A method for treating a subject exhibiting pulmonary arterial hypertension (PAH), comprising administering to the patient a therapeutically effective amount of an emulsion of claim 1.

10. A method for treating a subject exhibiting hypoxic pulmonary vasoconstriction (HPV), comprising administering to the patient a therapeutically effective amount of an emulsion of claim 1.

11. The method of claim 10, wherein the HPV is acute HPV, pulmonary hypertension, elevated pulmonary pressures, or high altitude pulmonary edema.

12. (canceled)

13. The method of claim 9, wherein the administering comprises intrapulmonary delivery of the emulsion.
